(19) United States
(12) Patent Application Publication
(10) Pub. No.: US 2014/0193455 A1
(43) Pub. Date: Jul. 10, 2014

(54) PHARMACEUTICAL COMPOSITION FOR PREVENTION OR TREATMENT OF STEROID-INDUCED DIABETES AND HEALTH FUNCTIONAL FOOD

(71) Applicant: Byoung Cheon KIM, Gimcheon-City (KR)

(72) Inventor: Byoung Cheon KIM, Gimcheon-City (KR)

(21) Appl. No.: 13/737,148

(22) Filed: Jan. 9, 2013

(51) Int. Cl. A61K 36/07

(52) U.S. Cl. CPC A61K 36/07
USPC 424/195.15

(57) ABSTRACT

A pharmaceutical composition for preventing or treating steroid-induced diabetes (SID) and a health functional food, and more particularly to a pharmaceutical composition for preventing or treating SID and a health functional food, contain a Ceriporia lacerata culture extract as an active ingredient. The pharmaceutical composition and the health functional composition have the effect of protecting INS-1 insulin-secreting cells by blocking the steroid-mediated proliferation inhibition and apoptosis induction of INS-1 insulin-secreting cells. Thus, the pharmaceutical composition and the health functional composition can be developed as the active ingredient of pharmaceutical or functional food compositions serving to protect or regenerate β-cells by blocking the steroid-mediated growth inhibition and apoptosis of β-cells.

### BACKGROUND OF THE INVENTION

1. Field of the Invention

The present invention relates to a pharmaceutical composition for preventing or treating steroid-induced diabetes (SID) and a health functional food, and more particularly to a pharmaceutical composition for preventing or treating SID and a health functional food, which contain a Ceriporia lacerata culture extract as an active ingredient.

2. Description of the Prior Art

Clinically, steroid-based drugs have strong anti-inflammatory and immune regulatory activities, and thus are frequently used as therapeutic agents against various human diseases, including rheumatoid arthritis, blood cancer, multiple sclerosis and nerve disorder, and in organ transplantation. However, many fundamental and clinical studies have reported that long-term or excessive use of steroid-based drugs causes various side effects in the human body. One of the side effects caused by steroid is steroid-induced diabetes (SID).

SID has a very close connection with a decrease in insulin secretion and synthesis, the induction of insulin resistance, and an increase in glucose production in the liver. SID is also known to have a deep connection with the steroid-mediated proliferation inhibition and apoptosis induction of β-cells (major insulin secretion and synthesis sites). However, the mechanisms of action for the steroid-mediated proliferation inhibition and apoptosis induction of β-cells have not yet been sufficiently identified. Therefore, for the prevention and treatment of SID, there is an urgent need not only for the identification of exact molecular mechanisms for the steroid-mediated proliferation inhibition and apoptosis induction of β-cells, but also for the investigation and development of β-cell protective (regenerating) substances capable of blocking the inhibition of proliferation and induction of apoptosis of β-cells caused by steroidal drugs.

Previous domestic studies reported that dexamethasone (Dex), a synthetic steroid drug, inhibits the proliferation and induces the apoptosis of insulin-secreting cells and other various kinds of cells. The effect of Dex on the induction of apoptosis was found to have a deep connection with the release of intracellular mitochondrial cytochrome c, an increase in caspase activity, the generation of reactive oxygen species, an increase in NF-κB transcription factor activity, a decrease in the phosphorylation (activation) of insulin receptor substrate-2 and protein kinase B(PKB), an increase in the expression of mitogen-activated protein kinase (MAPK) phosphatase-1 (MKP-1), and a decrease in the phosphorylation (activity) of extracellular signal-regulated protein kinase-1/2 (ERK-1/2).

Meanwhile, Ceriporia lacerata is a kind of white-rotting fungus and performs co-metabolism (lignin decomposition) in order to use carbon sources such as cellulose and hemi-cellulose in the ecosystem. The presence of Ceriporia lacerata was reported first in 2002, and for this reason, there have been only a very small number of studies on the industrialization of Ceriporia lacerata. With respect to the industrialization of Ceriporia lacerata, only two types of studies on the use of Ceriporia lacerata for soil contamination preventing and bleaching are known.

With respect to studies on the use of Ceriporia lacerata for foods and drugs, Korean Patent Registration No. 10-1031605 (entitled “method for preparing Ceriporia lacerata culture extract for prevention and treatment of diabetic disease and Ceriporia lacerata culture extract prepared thereby”) filed in the name of the present inventors is the only study in the world.

However, the effect of a Ceriporia lacerata mycelium culture on the blocking of the steroid-mediated proliferation inhibition and apoptosis induction of β-cells and the mechanism of action thereof have not yet been reported.

### SUMMARY OF THE INVENTION

Accordingly, the present invention has been made in order to solve the above-described problems occurring in the prior art, and it is an object of the present invention to provide a pharmaceutical composition for preventing or treating steroid-induced diabetes (SID) and a health functional food, which contain a Ceriporia lacerata culture extract as an active ingredient.

To achieve the above object, the present invention provides a pharmaceutical composition for preventing or treating steroid-induced diabetes (SID), which contains a Ceriporia lacerata culture extract as an active ingredient.

The present invention also provides a pharmaceutical composition for promoting survival of pancreatic β-cells, which contains a Ceriporia lacerata culture extract as an active ingredient.

The present invention also provides a pharmaceutical composition for inhibiting apoptosis of pancreatic β-cells, which contains a Ceriporia lacerata culture extract as an active ingredient.

The present invention also provides a health functional food for ameliorating steroid-induced diabetes (SID), which contains a Ceriporia lacerata culture extract as an active ingredient.

### BRIEF DESCRIPTION OF THE DRAWINGS

FIG. 1(A) shows the blocking effect of a Ceriporia lacerata culture extract (hereinafter also referred to as “CLCE”) on the steroid-mediated proliferation inhibition of INS-1 insulin-secreting cells.

FIG. 1(B) shows the blocking effect of a Ceriporia lacerata culture extract on the steroid-mediated apoptosis induction of INS-1 cells.

FIG. 2 shows the modulating effects of a Ceriporia lacerata culture extract on the expression and/or activation of MKP-1, ERK-1/2, and PKB in the steroid-treated INS-1 cells.

FIG. 3 shows LY294002 (PI3K/PKB inhibitor)-mediated elimination of the effects of a Ceriporia lacerata culture extract on the blocking of the steroid-mediated proliferation inhibition of INS-1 insulin-secreting cells.

FIG. 4(A) shows the effect of a Ceriporia lacerata culture extract on the decrease in survival rate of INS-1 caused by interleukin-1β (IL-1β).

FIG. 4(B) shows the effect of a Ceriporia lacerata culture extract on the decrease in survival rate of INS-1 caused by streptozotocin (STZ).

FIG. 4(C) shows the effect of a Ceriporia lacerata culture extract on the decrease in survival rate of INS-1 caused by thapsigargin (TG).

FIG. 4(D) shows the effect of a Ceriporia lacerata culture extract on the decrease in survival rate of INS-1 caused by tunicamycin (TN).

### DETAILED DESCRIPTION OF THE INVENTION

Hereinafter, the present invention will be described in detail.

The present inventors have made extensive efforts to develop a substance capable of suppressing or preventing the steroid-mediated proliferation inhibition and apoptosis induction of β-cells, and as a result, have experimentally demonstrated that a Ceriporia lacerata culture extract shows the effect of blocking both the steroid-mediated proliferation inhibition and apoptosis induction of INS-1 insulin-secreting cells, and this blocking effect has a deep connection with an increase in PKB activity, suggesting that this extract can also be used for the prevention or treatment of SID, thereby completing the present invention.

Therefore, the present invention provides a pharmaceutical composition for preventing or treating steroid-induced diabetes (SID), which contains a Ceriporia lacerata culture extract as an active ingredient.

The Ceriporia lacerata culture extract of the present invention may be used as a pharmaceutical composition for promoting survival of pancreatic β-cells or a pharmaceutical composition for inhibiting apoptosis of pancreatic β-cells.

The present invention also provides a health functional food for ameliorating steroid-induced diabetes (SID), which contains a Ceriporia lacerata culture extract as an active ingredient.

The Ceriporia lacerata culture extract that is the active ingredient of the inventive pharmaceutical composition and health functional food for preventing or treating steroid-induced diabetes (SID) can be prepared by a method comprising the steps of:

(a) culturing Ceriporia lacerata mycelia to obtain a Ceriporia lacerata mycelium culture;

(b) vacuum-drying or freeze-drying the culture to form powder;

(c) extracting the powder with one or more solvents selected from the group consisting of water, ethanol and methanol; and

(d) formulating the extract into a pharmaceutical composition and a health functional food.

The liquid culture of Ceriporia lacerata mycelia in step (a) is performed by culturing Ceriporia lacerata mycelia in liquid to obtain exopolysaccharide. A medium composition for the liquid culture may comprise 1-2 wt % of sugar, 0.2-1 wt % of glucose, 0.2-1 wt % of starch, 0.1-0.5 wt % of sorghum powder, 0.1-0.5 wt % of barley powder, 0.2-2 wt % of soy flour, 0.05-0.1 wt % of magnesium sulfate (MgSO₄), 0.05-0.1 wt % of monopotassium phosphate (KH₂PO₄), 0.05-0.1 wt % of dipotassium phosphate (K₂HPO₄) and 92-98 wt % of water.

Herein, the liquid culture is preferably carried out at a temperature of 20 to 25° C., a pH of 4.5-6.0, an illumination intensity of 0.5 LUX, an air injection rate of 0.5-1.5 kgf/cm² and a carbon dioxide concentration of 1,000-2,000 ppm for 8-13 days under a blue LED light source. Most preferably, the liquid culture is carried out under the conditions of temperature of ID, pH of 5, air injection rate of 1.0 kgf/cm² and carbon dioxide concentration of 1,500 ppm for 10 days, and in this case, the content of exopolysaccharide is the highest.

The parent strain that is used in step (a) is a strain obtained by culturing an excellent strain, stored in PDA medium at 4° C., in a shaking incubator at 25° C. for 7-9 days using PDB medium in an Erlenmeyer flask. Herein, the amount of the mycelium to be introduced as an inoculum source is most preferably 0.5% of the solution to be incubated. Because an increase in the mycelium amount (%/100 ml) does not lead to an increase in the content of exopolysaccharide, the medium composition should have selective culture conditions, which maximize the content of exopolysaccharide and are not the best nutrient ratio and environmental conditions for the growth of mycelia.

The culture solution is separated into mycelia and an aqueous solution. The separation is performed by removing mycelia from the culture solution using a centrifuge and repeatedly purifying the remaining solution using a multi-sheet filter press and a vibrating membrane separator (PALLSEP), followed by irradiation with UV rays for 1 minute. This is because the presence of mycelia in the culture solution results in the change in the content of the active ingredient due to the growth of the mycelia.

In step (b), the mycelium culture prepared in step (a) is vacuum-dried or freeze-dried to form powder. When the drying is carried out at high temperature, a significant portion of the active ingredient can be lost. For this reason, the drying is carried out at a temperature of 40° C. or lower, preferably 30° C. or lower, for 48-96 hours. In addition, for the drying in step (b), a vacuum freeze dryer is preferably used compared to a vacuum dryer in which a relatively high evaporation temperature is set, and in this case, the change in the content of the active substance is minimized.

In step (c), the dried mycelia culture obtained in step (b) is extracted with a solvent, thereby preparing a Ceriporia lacerata mycelium culture extract containing exopolysaccharide according to the present invention.

In step (c), 5 g of the dried powder is sufficiently suspended in 100 mL of distilled water, and the suspension is centrifuged at 8,000 rpm for 20 minutes. Cold alcohol is added to the supernatant in an amount corresponding to 2-3 times the amount of the supernatant, and the solution was placed in a refrigerator at 4° C. and allowed to stand for 12 hours.

The supernatant in the solution which had been allowed to stand is centrifuged again at 8,000 rpm for 20 minutes, and the precipitate is recovered, thereby preparing a crude exopolysaccharide. The extract is preferably vacuum-freeze-dried at 30° C. or lower.

In step (d), the solvent extract of the Ceriporia lacerata mycelium culture, obtained in step (c), is formulated into a pharmaceutical composition and a health functional food. In this process, the pharmaceutical composition and the health functional food further comprise, in addition to the extract, a suitable carrier, excipient or diluent which is generally used for the preparation of pharmaceutical compositions and health functional foods.

For example, a powder formulation is prepared by mixing 200 mg of the extract, 100 mg of rice powder and 10 mg of talc and packing the mixture into an airtight bag.

For example, a tablet formulation is prepared by mixing 100 mg of the extract, 50 mg of rice powder, 10 mg of lactose and 2 mg of magnesium stearate and compressing the mixture into a tablet.

For example, a liquid formulation is prepared by mixing 100 ml of the extract, 5 g of isomerized sugar, a suitable amount of pine fragrance and a suitable amount of a preservative and packing the mixture in a brown bottle. In this case, the material resulting from step (a) may be used instead of the extract.

The Ceriporia lacerata mycelium culture extract prepared according to the present invention as described above has significantly high contents of active ingredients effective for the treatment of steroid-induced diabetes, and thus has a very excellent effect of arresting and treating diabetes-related diseases and complications. More specifically, the Ceriporia lacerata mycelium culture extract according to the present invention contains exopolysaccharide, known to have anti-diabetic effects, in a very large amount of 3.00±0.03%/100 ml. In addition, the dried extract contains the exopolysaccharide in a very large amount of 5.00±0.02%/100 mg.

Hereinafter, the present invention will be described in further detail with reference to examples. It is to be understood, however, that these examples are for illustrative purposes and are not intended to limit the scope of the present invention.

### EXAMPLES

Materials and Methods

1. Preparation of Ceriporia lacerata Culture Extract

Ceriporia lacerata was isolated from Quercus serrata and subcultured to obtain a parent strain which was then freeze-stored at −80° C. The stored strain was subcultured 2-3 times in PDA medium (87 plastic bulbs), and then a sufficient amount of a complete strain was selected and stored in a refrigerator at 4° C. until use. In addition, 600 ml of PDB medium was placed in an Erlenmeyer flask, and then a PDA culture strain was added thereto and shake-cultured for 8 days. Also, a liquid culture medium comprising 1.5 wt % of sugar, 0.5 wt % of glucose, 0.5 wt % of potato starch, 0.25 wt % of wheat powder, 0.25 wt % of sorghum powder, 0.05 wt % of magnesium sulfate (MgSO₄), 0.05 wt % of monopotassium phosphate (KH₂PO₄), 0.05 wt % of dipotassium phosphate and 96.85 wt % of water was sterilized in a 800-L fermenter at 121° C. and at an air injection rate of 1.5 kgf/cm², and then cooled to 23° C., after which it was inoculated with 600 ml of the PBD culture strain to be used as a starter. Then, Ceriporia lacerata mycelia were liquid-cultured in the medium at a temperature of 23° C., an aeration rate of 0.5-1.5 kgf/cm² and a carbon dioxide concentration of 1,000-2,000 ppm for 10 days, thereby preparing a Ceriporia lacerata mycelium culture.

The prepared Ceriporia lacerata mycelium culture was freeze-dried using a vacuum freeze dryer at a temperature of 25° C. for 72 hours to form powder. Five g of the dry powder was suspended sufficiently in 100 ml of distilled water and then centrifuged at 8,000 rpm for 20 minutes, and cold alcohol was added to the supernatant in an amount corresponding to 2-3 times the amount of the supernatant. The resulting solution was placed in a refrigerator at 4° C. and allowed to stand for 12 hours. The supernatant in the solution which had been allowed to stand was centrifuged again at 8,000 rpm for 20 minutes, and the precipitate was recovered, thereby extracting a crude exopolysaccharide. The crude exopolysaccharide was dried in a freeze dryer for 72 hours, thereby obtaining a complete exopolysaccharide.

2. Experimental Materials

RPMI-1640 medium, fetal bovine serum, penicillin and streptomycin were purchased from WelGENE (Daegu, Korea). IL-1β was purchased from R&D SYSTEMS (Minneapolis, Minn., USA). p-ERK-1/2, T-ERK-1/2, p-PKB and T-PKB antibodies were purchased from Cell Signaling Technology (Danvers, Mass., USA). LY294002 and streptozotocin (STZ) were purchased from Biomol (Plymouth Meeting, Pa., USA). Antibodies of MKP-1 and goat anti-rabbit or anti-mouse secondary horseradish peroxidase were purchased from Santa Cruz Biotechnology (Santa Cruz, Calif., USA). Bradford reagent was purchased from Bio-Rad (Hercules, Calif., USA). Enzyme-linked chemiluminescence (ECL) Western blot reagent was purchased from Thermo SCIENTIFIC (Waltham, Mass., USA). Dexamethasone (Dex), actin antibody and other reagents were purchased from Sigma (St. Louis, Mo., USA).

3. Cell Culture

The rat insulin-secreting cell line INS-1 was maintained in RPMI-1640 medium containing 10% heat-inactivated FBS (fetal bovine serum), 100 units/ml of penicillin and 100 μg/ml of streptomycin at 37° C. under the conditions of 95% humidity and 5% CO₂.

4. Cell Counting Analysis

One day before treatment with reagent or CLCE, INS-1 cells were cultured in a 24-well plate at a concentration of 1×10⁵ cells/well in a volume of 500 μL. The INS-1 cells were treated with the indicated concentrations of reagent (Dex, LY294002, IL-1β, STZ, TG or TN) and/or CLCE for the indicated time. At each point of time, the cells were stained with trypan blue dye, and the living INS-1 cells were counted under a microscope. The cell counting was repeated three times. Data were expressed as the mean of three independent experiments.

5. Measurement of DNA Fragmentation

One day before treatment with reagent or CLCE, INS-1 cells were cultured in a 6-well plate at a concentration of 1×10⁶ cells/well in a volume of 2 mL. The INS-1 cells were incubated with the indicated concentrations of Dex and/or CLCE for 24 hours. After 24 hours, each of the cells was collected, washed and lysed in buffer (50 mM Tris (pH 8.0), 0.5% sarkosyl, 0.5 mg/mL proteinase K, and 1 mM EDTA) at 55° C. for 3 hours, and RNase A (0.5 g/mL) was added thereto, followed by additional incubation at 55° C. for 18 hours. The lysates were centrifuged at 10,000× g for 20 minutes. Genomic DNA in the supernatant was extracted with the same volume of a mixture of phenol/chloroform/isoamyl alcohol mixture (25:24:1) and analyzed by electrophoresis on 1.7% agarose gel. The DNA was stained with ethidium bromide (0.1 g/mL), visualized by UV irradiation and then photographed.

6. Preparation of Whole Cell Lysate

One day before treatment with reagent or CLCE, INS-1 cells were cultured in a 6-well plate at a concentration of 0.5×10⁶ cells/well in a volume of 2 mL. These INS-1 cells were incubated with the indicated concentrations of Dex and/or CLCE for 4 hours. Then, the INS-1 cells were washed twice with PBS supplemented with 1 mM Na₃VO₄ and 1 mM NaF, and the washed cells were exposed to cell lysis buffer (50 mM Tris-Cl (pH 7.4), 150 mM NaCl, 0.1% sodium dodecyl sulfate, 0.25% sodium deoxycholate, 1% Triton X-100, 1% Nonidet P-40, 1 mM EDTA, 1 mM EGTA, proteinase inhibitor cocktail (1×)). The cell lysate was collected in a 1.5-mL tube and centrifuged at 12,000 rpm at 4° C. for 20 minutes. The supernatant was collected and the protein concentration thereof was determined with Bradford reagent.

7. Western Blot Analysis

Fifty μg of protein was separated by 10% SDS-PAGE and transferred onto a nitrocellulose membrane (Millipore). The membrane was washed with TBS (10 mM Tris, 150 mM NaCl) [TBST] supplemented with 0.05% (vol/vol) Tween 20 and was blocked with TBST containing 5% (wt/vol) non-fat dry milk. The membrane was incubated with antibody specific to MKP-1 (1:2,000), p-ERK-1/2 (1:2,000), T-ERK-1/2 (1:2,000), p-PKB (1:2,000), T-PKB (1:2,000) or actin (1:5,000) overnight at 4° C. Then, the membrane was exposed to horseradish peroxidase-conjugated secondary antibody. Then, the membrane was washed with TBST at room temperature. Immune reactivity was detected with ECL reagent. Equal protein loading was evaluated by the expression level of actin protein.

Experimental Results

1. Blocking of the Decrease in Survival and Induction of Apoptosis of INS-1 Cells Caused by Steroid Dex

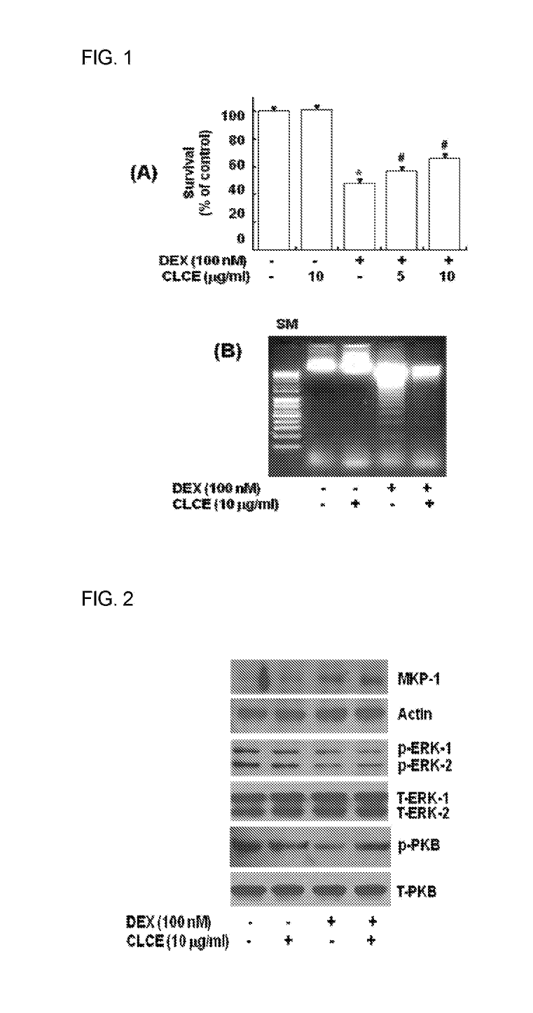

The effects of different concentrations of CLCE on the decrease in survival and induction of apoptosis of INS-1 cells caused by steroid Dex were measured by cell counting analysis and nuclear DNA fragmentation, respectively. As can be seen in panel A of FIG. 1, when INS-1 cells were treated with Dex alone for 24 hours, the survival rate of the INS-1 cells decreased by about 50%, but when INS-1 cells were treated with a combination of Dex and CLCE, the decrease in survival rate of INS-1 cells by Dex was greatly blocked. Then, the effect of CLCE on the apoptosis of INS-1 cells caused by Dex was determined by measuring nuclear DNA fragmentation known as an apoptosis marker. As can be seen in panel B of FIG. 1, when INS-1 cells were treated with Dex alone for 24 hours, the nuclear DNA fragmentation of the cells increased. However, when INS-1 cells were treated with a combination of Dex and CLCE, Dex-induced DNA fragmentation in the INS-1 cells did not occur. Such results clearly show that CLCE has an INS-1 cell protective effect of blocking the decrease in survival and induction of apoptosis of INS-1 cells caused by Dex.

2. Selective Blocking of Dex-Induced Decrease in PKB Activity in INS-1 Cells

The activation of ERK-1/2 and/or PKB has a close connection with the survival of cells.

MKP-1 is a kind of phosphatase and interferes with the phosphorylation of ERK-1/2 and/or PKB. Thus, the present inventors measured whether treatment of INS-1 cells with Dex and/or CLCE regulates the expression and activation (phosphorylation) of these proteins. As can be seen in FIG. 2, when INS-1 cells were treated with Dex alone for 4 hours, the expression of MKP-1 protein greatly increased, but the phosphorylation of ERK-1/2 and PKB greatly decreased. When INS-1 cells were treated with a combination of Dex and CLCE, the Dex-induced decrease in the phosphorylation of PKB was greatly blocked, but the Dex-induced increase in the expression of MKP-1 protein and the Dex-induced decrease in the phosphorylation of ERK-1/2 were not influenced by CLCE treatment. Such results clearly show that CLCE has the effect of specifically blocking the Dex-induced decrease in the phosphorylation of PKB in INS-1 cells, and suggest that this CLCE-mediated increase in the phosphorylation of PKB occurs regardless of the expression of MKP-1.

3. Effect on Blocking of Dex-Induced Decrease in Survival of INS-1 Cells and Relationship with PKB Activation (Phosphorylation)

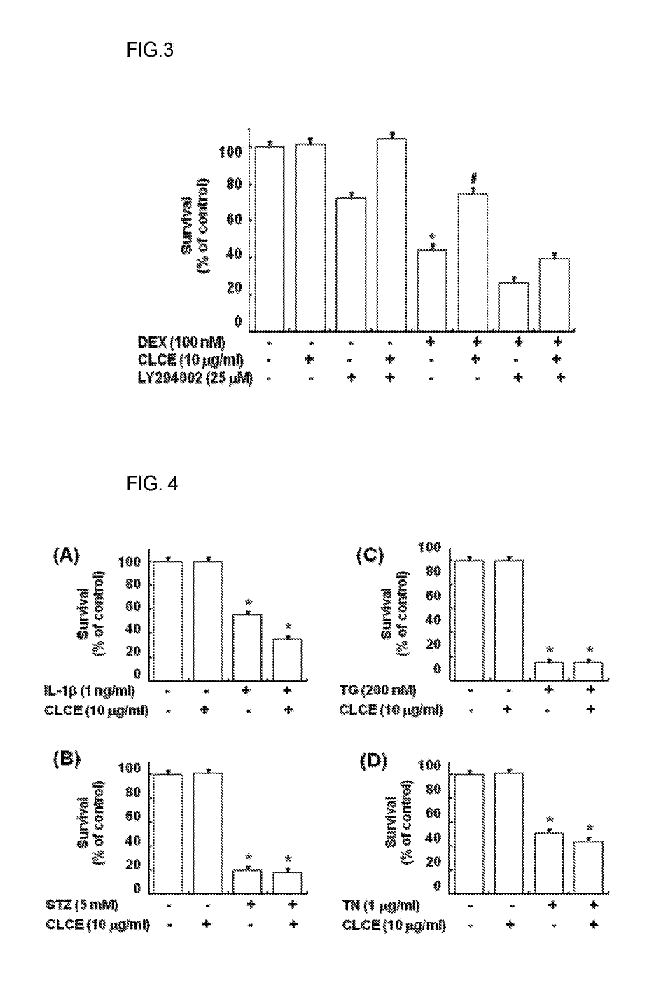

In order to examine the importance of PKB activation in the effect of CLCE on the blocking of the Dex-induced decrease in the survival of INS-1 cells, the present inventors performed the experiment using LY294002 (PI3K/PKB inhibitor). As can be seen in FIG. 3, when INS-1 cells were treated with LY294002 alone, the survival of the INS-1 cells decreased by about 24% compared to that of the control. Meanwhile, when INS-1 cells were treated with Dex alone, the cell survival decreased by about 55%, but when INS-1 cells were treated with a combination of Dex and LY294002, the degree of the decrease in the survival of the INS-1 cells was 75% higher than the decrease (55%) in INS-1 cell survival caused by Dex alone. When INS-1 cells were treated with CLCE alone, the survival of the INS-1 cells did not change, and when INS-1 cells were treated with a combination of LY294002 and CLCE, the survival of the INS-1 cells was slightly higher than that of the control. It was observed that, when INS-1 cells were treated with a combination of Dex and CLCE, the INS-1 cell survival decreased by about 22% compared to the decrease (55%) in INS-1 cell survival caused by Dex alone. In addition, when INS-1 cells were treated with a combination of Dex, CLCE and LY294002, the cell survival decreased by about 61% compared to the decrease (22%) in INS-1 cell survival caused by treatment with the combination of Dex and CLCE. Such results clearly show that PKB activity (maintained or increased) is very important for the survival of INS-1 cells and that Dex reduces the survival of INS-1 cells by inhibiting PKB activity. In addition, such results suggest that maintaining and increasing PKB activity by CLCE is a very important mechanism for blocking the Dex-mediated decrease in the survival of INS-1 cells.

4. Effect on the Decrease in INS-1 Cell Survival Caused by IL-1β, STZ, TG or TN

In order to examine whether the INS-1 cell protective effect of CLCE appears for β-cell toxic substances other than steroid (Dex), INS-1 cells were treated with IL-1β, STZ, TG or TN alone or in combination with CLCE for 24 hours, and then the effect of CLCE on the protection of INS-1 cells was examined. As can be seen in panels A, B, C and D of FIG. 4, treatment of INS-1 cells with IL-1β, STZ, TG or TN showed decreases in INS-1 cell survival of about 45%, 82%, 88% and 50% compared to the control. However, treatment of INS-1 cells with IL-1β, STZ, TG or TN in combination with CLCE showed decreases in INS-1 cell survival of about 65%, 81%, 88% and 58%. Such results suggest that CLCE has the effect of specifically blocking the Dex-induced decrease in the survival of INS-1 cells.

As described above, the inventive pharmaceutical composition and health functional composition have the effect of protecting INS-1 insulin-secreting cells by blocking the steroid-mediated proliferation inhibition and apoptosis induction of INS-1 insulin-secreting cells. Thus, the inventive pharmaceutical composition and health functional composition can be developed as the active ingredient of pharmaceutical or functional food compositions serving to protect or regenerate β-cells by blocking the steroid-mediated growth inhibition and apoptosis of β-cells.

1. A pharmaceutical method for preventing or treating steroid-induced diabetes (SID), comprising a step for using a composition including a Ceriporia lacerata culture extract as an active ingredient.

2. A pharmaceutical method for promoting survival of pancreatic β-cells, comprising a step for using a composition including a Ceriporia lacerata culture extract as an active ingredient.

3. A pharmaceutical method for inhibiting apoptosis of pancreatic β-cells, comprising a step for using a composition including a Ceriporia lacerata culture extract as an active ingredient.

4. A method for ameliorating steroid-induced diabetes (SID), comprising a step for using a health functional food including a Ceriporia lacerata culture extract as an active ingredient.
